(19) United States
(12) Patent Application Publication
(10) Pub. No.: US 2022/0387215 A1
(43) Pub. Date: Dec. 8, 2022

(54) DEVICE AND METHOD FOR OCULAR SURGERY

(71) Applicant: MICROSURGICAL TECHNOLOGY, Redmond, WA (US)

(72) Inventors: Dominic A. Filice, Seattle, WA (US); Teresa F. Lumini, Bellevue, WA (US); Lyle E. Erman, Redmond, WA (US)

(21) Appl. No.: 17/124,434

(22) Filed: Dec. 16, 2020

Related U.S. Application Data
(60) Provisional application No. 62/948,733, filed on Dec. 16, 2019.

(51) Int. Cl. A61F 9/007; A61B 10/02

(52) U.S. Cl. CPC A61F 9/00763; A61B 10/0275; A61F 9/00754

(57) ABSTRACT

Provided are embodiments of systems, devices and methods to aid in or perform the capsulotomy procedure via access into an eye through a small incision. Embodiments of the present disclosure may employ the unique characteristics of shape-memory materials to enable device access into the eye through the requisite small incision size, and via simple mechanical means either to create a template for surgeons to follow in order to create an appropriately-sized capsulotomy, or to create such a capsulotomy via cutting, tearing, or abrading the lens capsule.

### CROSS REFERENCE TO RELATED APPLICATION

The present application claims priority to U.S. Provisional Patent Application No. 62/948,733, filed Dec. 16, 2019, the disclosure of which is hereby incorporated by reference in its entirety for all purposes.

### FIELD

The subject matter described herein relates generally to systems, methods, and devices for ocular surgery, and particularly for capsulotomy.

### BACKGROUND

The capsulotomy is an early and critical part of cataract surgery in which a circular incision is made in the anterior capsule of the eye's lens. Its purpose is to enable access to the cataractous lens for extraction and later for anchoring/centering an intraocular lens (IOL). There are a number of potential complications that can arise when performing the capsulotomy, including: 1) creating too small a capsulotomy, which increases the complexity of cataract removal; 2) creating too large a capsulotomy, which reduces the capsule's ability to retain an IOL and can introduce post-operative optical complications such as posterior capsular opacification (PCO), IOL tilt, IOL decentration, and lead to a less effective IOL position; 3) creating a radialized capsulotomy, which increases the complexity of cataract removal and the potential for vitreous prolapse into the anterior segment; and 4) creating a discontinuous or non-circular capsulotomy, which limits surgeon's comfort in manipulating the lens due to concerns of creating capsulotomy tears.

In addition to the potential complications that can arise when creating the capsulotomy, there are a number of problematic scenarios that at the outset increase the difficulty of the procedure and the risk of complications occurring, including: 1) the presence of zonular instability, such as zonular weakness, missing zonules, or zonules that are too long as in Marfan Syndrome patients, which all can lead to a lack of lens capsule stability during the capsulotomy; 2) white intumescent cataract cases, where the capsule is prone to tear out radially once punctured due to higher than normal intracapsular pressure as a result of cortex liquefaction instead of cortex solidification; and 3) young patients, which have highly elastic capsules that are difficult to puncture.

Current methods to perform an anterior capsulotomy include manually creating the capsulotomy using a cystatome (i.e., a bent needle), manually creating the capsulotomy using forceps, or employing powered systems that automatically create the capsulotomy.

Current powered systems include: 1) the Zepto device by Mynosys Cellular Devices, which utilizes suction and the energizing of a metal ring to thermally cause a phase change in water molecules at the ring-to-capsule contact area and cleave the capsule; 2) the Aperture CTC device by International BioMedical Devices, which utilizes the energizing of a metal ring to thermally cut the capsule; 3) the CAPSULaser device by EXCEL-LENS Inc., which utilizes a continuous laser to thermally cut the capsule after it has been stained with a special Trypan blue formulation that selectively absorbs the continuous laser energy; and 4) numerous femtosecond laser systems, which utilize ultra-short laser pulses to thermally ablate tissue focused in the laser path. The price and complexity of the aforementioned methods/devices vary considerably, and each have their own pros and cons.

Thus, there exists a need for a simple, cost effective, and reliable method and device to aid in or perform the capsulotomy procedure.

### SUMMARY

Provided herein are example embodiments of cost effective and reliable systems, devices and methods to aid in or perform the capsulotomy procedure. Furthermore, with the trend towards minimally invasive surgeries, systems, devices and methods can aid in or perform the capsulotomy via access into an eye through a small incision, for example of 2.2 mm or less. In some embodiments, to meet these needs and give confidence to surgeons performing the capsulotomy, embodiments of the present disclosure may employ the unique characteristics of shape-memory materials to enable device access into the eye through the requisite small incision size, and via simple mechanical means either to create a template circle for surgeons to follow in order to create a circular and appropriately-sized capsulotomy, or to create such a capsulotomy via cutting, tearing, or abrading the lens capsule.

In some embodiments, the present disclosure may include a capsulotomy device comprising: an outer shell, a cannula housed within the outer shell, having proximal and distal ends, a shape-memory filament housed within the cannula and extendable through the distal end of the cannula, the shape-memory filament having distal and proximal ends, wherein the shape-memory filament has a bend at the shape-memory filament's distal end; and an actuator located on the outer shell and operably coupled to the shape-memory filament to cause the filament to extend and retract to and from the cannula's distal end.

In some embodiments, the present disclosure may include a capsulotomy device comprising: a cannula having proximal and distal ends, a shape-memory element housed within the cannula and extendable through the distal end of the cannula, the shape-memory element having distal and proximal ends, wherein the shape memory wire has a bend at the shape memory wire's distal end, and an actuator operably coupled to the shape memory wire to cause said wire to extend and retract to and from the cannula's distal end.

In some embodiments, the present disclosure may include a capsulotomy method comprising: introducing a cannula containing a shape-memory element into an anterior chamber of an eye, extending the shape-memory element out of the cannula, placing the shape-memory element into contact with a lens capsule of the eye, centering the shape-memory element on the lens capsule, and while maintaining contact between the shape-memory element and the lens capsule, retracting the shape-memory element into the cannula.

This summary and the following detailed description are merely exemplary, illustrative, and explanatory, and are not intended to limit, but to provide further explanation of the invention as claimed. Additional features and advantages of the invention will be set forth in the descriptions that follow, and in part will be apparent from the description, or may be learned by practice of the invention. The objectives and other advantages of the invention will be realized and attained by the structure particularly pointed out in the written description, claims and the appended drawings.

### BRIEF DESCRIPTION OF THE DRAWINGS

The present invention may be better understood by referring to the following figures. The components in the figures are not necessarily to scale. Emphasis instead being placed upon illustrating the principles of the disclosure. In the figures, reference numerals designate corresponding parts throughout the different views.

FIG. 1 illustrates an exemplary anatomy of an eye.

FIGS. 2A to 2F illustrate exemplary overall configuration of a portion of a capsulotomy device, according to some embodiments of the present disclosure.

FIGS. 3A to 3H illustrate exemplary variations of the shape-memory wire element(s), according to some embodiments of the present disclosure.

FIGS. 4A to 4AI illustrate cross-sectional views of various exemplary shape-memory elements, according to some embodiments of the present disclosure.

FIGS. 5A to 5K illustrate side views of additional exemplary, non-limiting variations of shape-memory elements, according to some embodiments of the present disclosure.

FIG. 6 illustrates an exemplary operation of a shape-memory element device, according to some embodiments of the present disclosure.

FIG. 7 illustrates an exemplary perspective view of a shape-memory element being placed against a lens capsule, according to some applications of the present disclosure.

FIGS. 8A to 8E illustrate additional variations of wire-element devices, according to some embodiments of the present disclosure.

FIGS. 9A to 9N illustrate examples of shape-memory element devices reduced to practice, according to some embodiments of the present disclosure.

FIGS. 10A to 10F illustrate various views of an exemplary capsulotomy shape-memory device reduced to practice, according to some embodiments of the present disclosure.

FIG. 11 illustrates an exemplary rotating cutting element, according to some embodiments of the present disclosure.

### DETAILED DESCRIPTIONS

The following disclosure describes various embodiments of the present invention and method of use in at least one of its preferred, best mode embodiments, which is further defined in detail in the following description. Those having ordinary skill in the art may be able to make alterations and modifications to what is described herein without departing from its spirit and scope. While this invention is susceptible to different embodiments in different forms, there is shown in the drawings and will herein be described in detail a preferred embodiment of the invention with the understanding that the present disclosure is to be considered as an exemplification of the principles of the invention and is not intended to limit the broad aspect of the invention to the embodiment illustrated. All features, elements, components, functions, and steps described with respect to any embodiment provided herein are intended to be freely combinable and substitutable with those from any other embodiment unless otherwise stated. Therefore, it should be understood that what is illustrated is set forth only for the purposes of example and should not be taken as a limitation on the scope of the present invention.

As used herein and in the appended claims, the singular forms “a,” “an,” and “the” include plural referents unless the context clearly dictates otherwise.

In general, terms such as “coupled to,” and “configured for coupling to,” and “secure to,” and “configured for securing to” and “in communication with” (for example, a first component is “coupled to” or “is configured for coupling to” or is “configured for securing to” or is “in communication with” a second component) are used herein to indicate a structural, functional, mechanical, electrical, signal, optical, magnetic, electromagnetic, ionic or fluidic relationship between two or more components or elements. As such, the fact that one component is said to be in communication with a second component is not intended to exclude the possibility that additional components may be present between, and/or operatively associated or engaged with, the first and second components.

In the following description and in the figures, like elements are identified with like reference numerals. The use of “e.g.,” “etc.,” and “or” indicates non-exclusive alternatives without limitation, unless otherwise noted. The use of “including” or “includes” means “including, but not limited to,” or “includes, but not limited to,” unless otherwise noted.

As used herein, the term “and/or” placed between a first entity and a second entity means one of (1) the first entity, (2) the second entity, and (3) the first entity and the second entity. Multiple entities listed with “and/or” should be construed in the same manner, i.e., “one or more” of the entities so conjoined. Other entities may optionally be present other than the entities specifically identified by the “and/or” clause, whether related or unrelated to those entities specifically identified. Thus, as a non-limiting example, a reference to “A and/or B,” when used in conjunction with open-ended language such as “comprising” can refer, in one embodiment, to A only (optionally including entities other than B); in another embodiment, to B only (optionally including entities other than A); in yet another embodiment, to both A and B (optionally including other entities). These entities may refer to elements, actions, structures, steps, operations, values, and the like.

Provided herein are example embodiments of cost effective and reliable systems, devices and methods to aid in or perform the capsulotomy procedure.

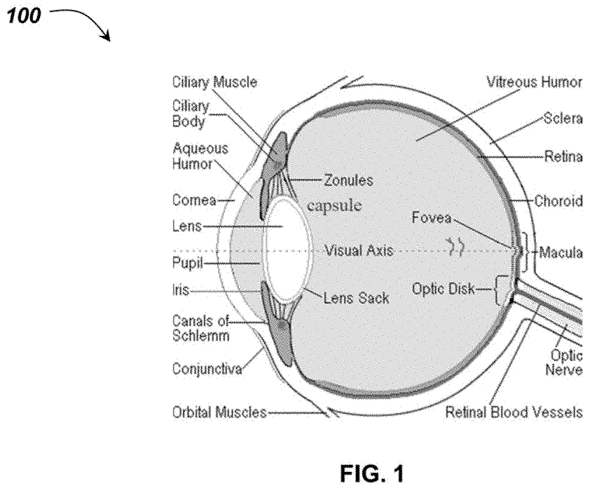

To help in the descriptions herein, FIG. 1 illustrates an anatomy of an eye 100 and illustrates terminology known in the art. As noted in the figure, the eye includes a cornea, lens, pupil, iris, canals of Schlemm, conjunctiva, orbital muscles, ciliary muscle ciliary body, aqueous humor, zonules, lens capsule, fovea, optic disk, vitreous humor, sclera, retina, choroid, macula, optic nerve, retinal blood vessels, wherein the cornea, lens, and pupil form a visual axis.

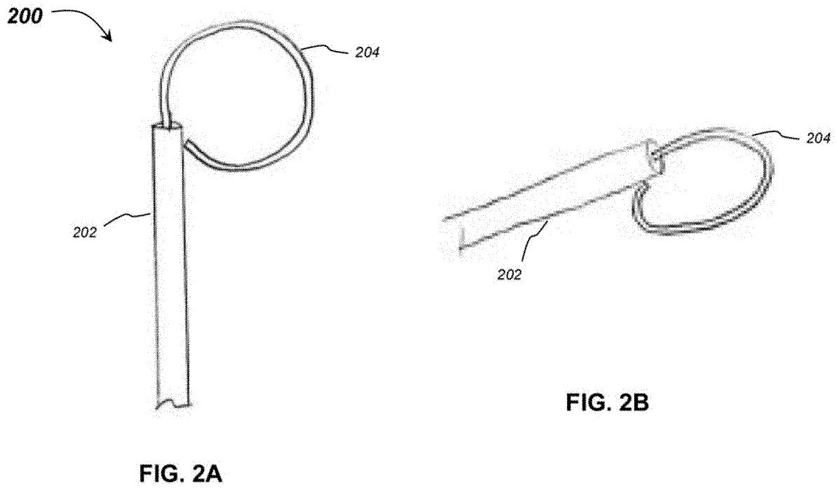

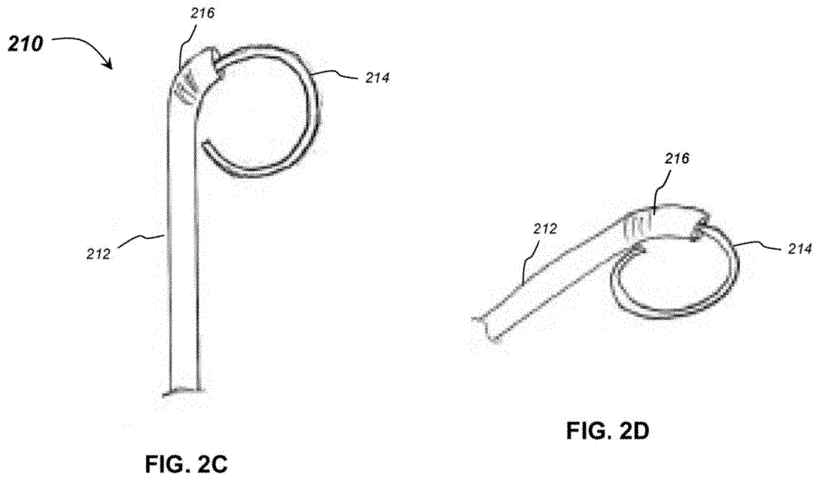

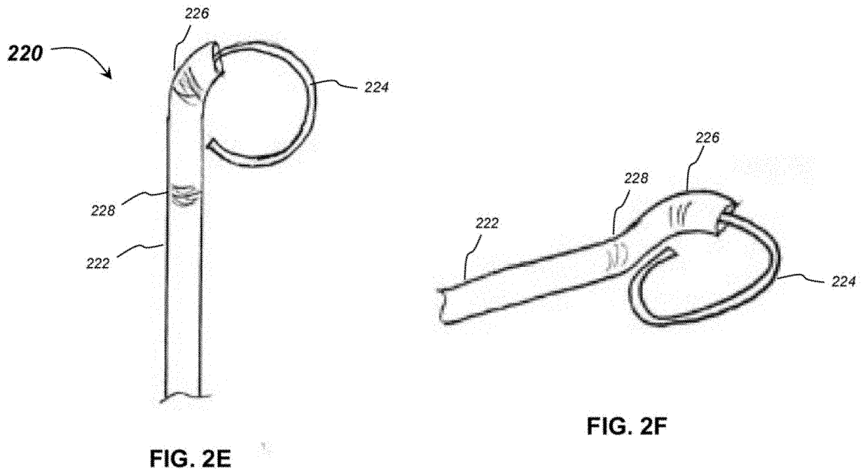

Generally, a capsulotomy device of the present disclosure may include a handle, an actuator, and a proximal end portion. The proximal portion (illustrated, for example, in FIGS. 2A to 2F) may include a cannula and a shape-memory element. In some embodiments of the present disclosure, the cannula may have a cross-sectional circular shape, oval shape, square shape, or any shape profile. In some embodiments, the cannula of the present disclosure may be straight. In some embodiments, the cannula may include one bend (at/near distal end, about 2.75 mm radius, to help ensure the shape-memory element stays in-plane with the bend and adds an element of control). In some embodiments, the cannula may include two bends (proximal to distal end, about 11 degrees (0-to-30-degree range), to help ensure the shape-memory element lays flat against the lens capsule based on the geometry/anatomy of the eye and the typical incision location; stated another way, this bend can help get the device past the iris and down fully against the lens capsule). In some embodiments, the cannula may include more bends.

In some embodiments, the cannula may be made of steel, stainless steel, plastic, composite, etc., and preferably of a biocompatible material. The cannula may be made drawn, molded, extruded, formed, etc.

In some embodiments, the size of the cannula may be 17 gauge, 19 gauge, 20 gauge, 21 gauge, 22 gauge, 23 gauge, 24 gauge, 25 gauge, 26 gauge, 27 gauge, 28 gauge, 29 gauge, or of any wall thickness designation. Alternatively, the cannula may be sized between 0.3 mm to 2.2 mm in any cross-sectional outer dimension.

In some embodiments, the cannula may have a distal end that may be ground or polished, for example, to remove any/all burrs or irregularities that could catch on and/or tear tissue. The distal end may be flared or shaped, for example, to accept variations of shape-memory element(s) and/or their distal end(s). The distal end may be over-molded, for example, to accept and guard variations of shape-memory element(s) and/or their distal end(s).

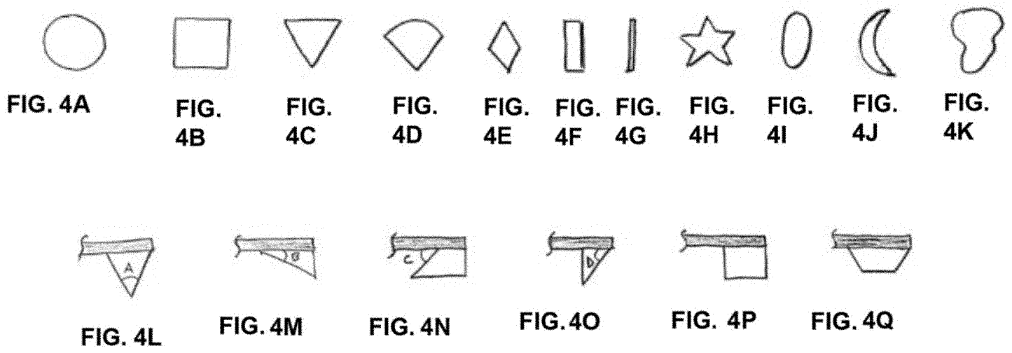

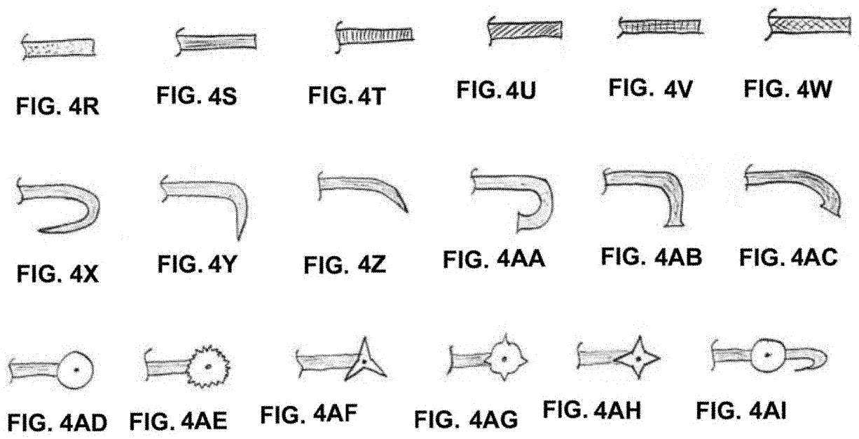

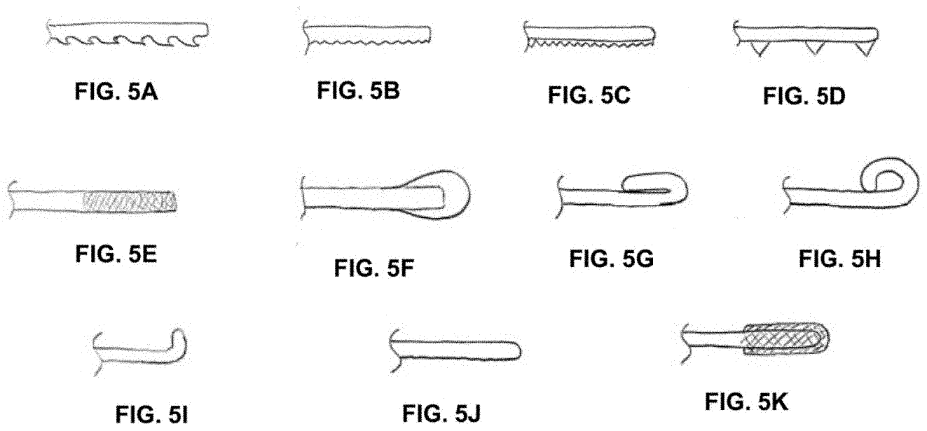

In some embodiments, the capsulotomy device of the present disclosure may include cutting/tearing element(s) (e.g., in the form of separate blade(s)), which are shown further in FIGS. 4A to 4AI, and FIGS. 5A to 5K). In some embodiments, the device may include none (e.g., in embodiments wherein the surface of the shape-memory element is abrasive), one, two, three, or any number of cutting/tearing elements.

The cutting/tearing element(s) may be located at the distal end of a shape-memory element, or any location on the shape-memory element with variable or regular spacing when two or more are utilized.

The cutting/tearing element(s) may be made of metal, carbon steel, stainless steel, titanium, any metal or steel alloy, plastic, ruby, sapphire, diamond, any gem, glass, obsidian, volcanic glass, ceramic, composite, etc.

The cutting/tearing element(s) may have any orientation (towards the lens capsule, at an angle to the lens capsule, etc.).

The cutting/tearing element(s) may have any joining method to join/adhere, examples include welding, sonic welding, soldering, gluing/epoxying, etc. In some embodiments, biocompatible method may be preferred.

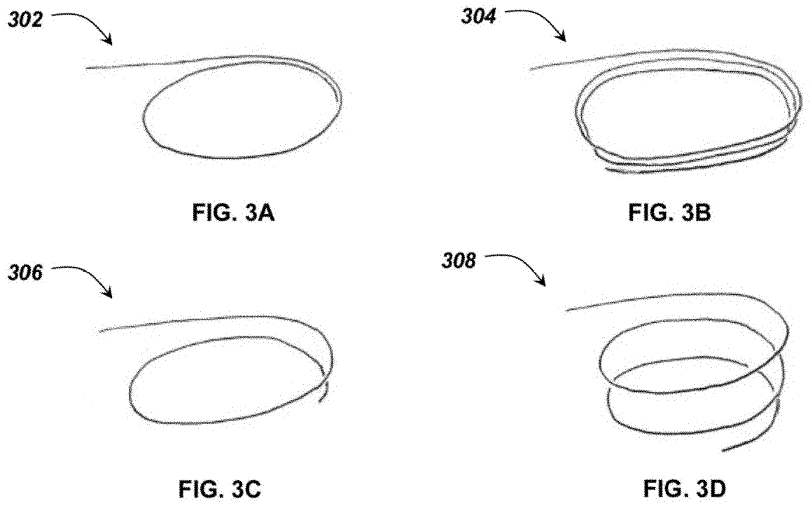

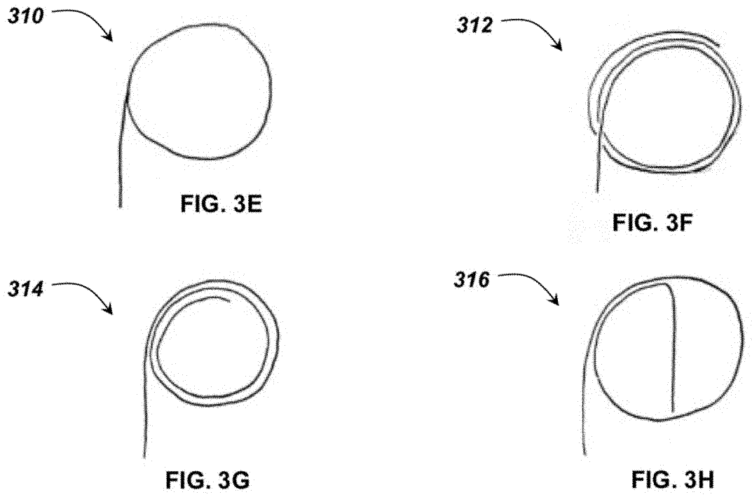

In some embodiments, the capsulotomy device of the present disclosure may include shape-memory element(s) and shown further in FIGS. 3A to 3H, FIGS. 4A to 4AI, and FIGS. 5A to 5K. The shape-memory element may have different cross-sectional shape. In some embodiments, inherent characteristics of the cross-sectional shape may be utilized for creating a capsulotomy template or the capsulotomy itself. For example, the “sharp” edge of a shape-memory element with a pie-shaped cross-section may be used to create the template/capsulotomy. The shape-memory element may be solid or hollow.

The shape-memory element may have an outer diameter of 0.006″, 0.008″, 0.010″, 0.012″, 0.014″, 0.015″, or any diameter that can slidably fit inside the paired cannula. Alternatively, the shape-memory element may be sized between 0.1 mm to 2.1 mm in any cross-sectional outer dimension.

The surface of the shape-memory element may be smooth/in as-drawn condition, sharpened on one edge, sharpened on two edges, sharpened on several edges, roughened through filing, roughened through sandblasting, roughened through sanding, roughened through tumbling with abrasives, roughened/grooved through pressing, or given an abrasive surface via adhering an abrasive compound (e.g., diamond dust), etc.

The shape-memory element may be made of shape-memory alloy (e.g., nitinol), shape-memory polymer, suture material (e.g., nylon, polypropylene), super-elastic material, steel, stainless steel, steel alloy, plastic, composite, etc.

The shape-memory element may have a circular shape, oval shape, or any amorphous shape. In the example case of nitinol material, the shape of the shape-memory element may be set by fixturing and heat treating. In some embodiments, the shape memory element may be formed to have a circular distal portion and an essentially straight proximal portion (the straight portion may interface with an actuator or handpiece). In some embodiments, the circular distal portion may have a 2.75 mm radius (but can vary from 2 mm to 3.25 mm). The circular distal portion may be formed into a coiled configuration such that a complete circle is created when viewed from the top, and it may have a pitch or minimal pitch (e.g., pitch of 1 mm, pitch of 0.5 mm, or essentially no pitch), and may wrap around greater than 360 degrees (e.g., greater than 360 degrees, approximately 370 degrees, approximately 730 degrees, approximately 1090 degrees, etc.). In some embodiments, the shape-memory element may take the form of an outward spiral (with no pitch or with pitch), an inward spiral (with no pitch or with pitch), or a cross-arm configuration.

In some embodiments, the distal end may be in as-drawn condition, ground or polished smooth (e.g., to prevent inadvertent puncturing or piercing of tissue), given a sharp pencil-point tip, flattened and sharpened, bent at any angle relative to the longitudinal axis of the shape-memory element (less than, at, or greater than 90 degrees), capped with a separate structural element (to either protect the end from causing any potential damage to tissue, or to instead purposely be used to create the template/capsulotomy), covered in an epoxy/adhesive or other means to encapsulate the end (to prevent inadvertent puncturing or piercing of tissue), made with means to accept a rotatable cutting element, or any combination of the above.

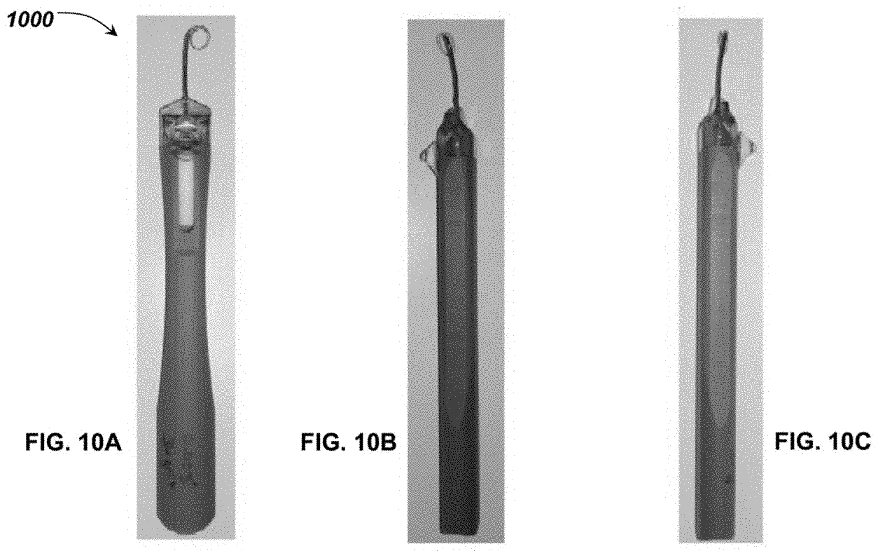

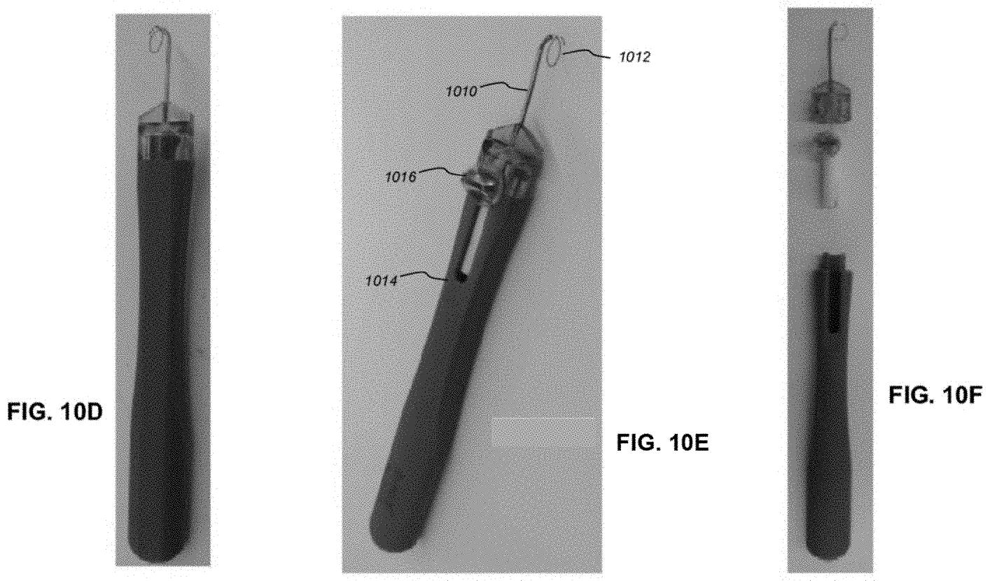

In some embodiments, the capsulotomy device of the present disclosure may include an actuator, which is shown further in FIGS. 10A to 10F. The actuator may include a button or a slide to actuate and cause the filament to extend and retract through the distal end of the cannula. In some embodiments, the actuator may simply operate to pull and push the proximal portion of the shape-memory element through the cannula. It may include a thumb button or slider or roller that moves within a shaft/handpiece. It may be spring-loaded (e.g., to extend or retract the shape-memory element quickly upon actuation). It may be geared or otherwise provided with a mechanical advantage or may be completely manually operated.

In some embodiments, the capsulotomy device of the present disclosure may also include a handpiece or shaft, which is shown further in FIGS. 10A to 10F. In some embodiments, the handpiece or shaft may be elongate and house the cannula and the shape-memory elements and the actuator. The handpiece or shaft may enable a user to extend and retract the shape-memory element from the cannula. The outer shell of the handpiece or shaft may isolate the cannula and the shape-memory element from the user's hand to maintain sterile environment.

Turning now to the drawings, FIGS. 2A to 2F illustrate exemplary overall configuration of a portion of a capsulotomy device, according to some embodiments. The proximal portion is illustrated in FIGS. 2A to 2F without showing the handle or actuators. In some embodiments, the portion may include a cannula and a shape-memory element. The shape-memory element may be a wire (filament) element.

FIG. 2A shows a top view of an exemplary portion 200, according to some embodiments. FIG. 2B shows a perspective view of the portion 200. In some embodiments, the portion 200 may include a cannula 202 and a shape-memory element 204, wherein the cannula 202 may not have a bend. In some embodiments, the shape-memory element 204 may have an open circular shape.

FIGS. 2C and 2D show a top view and a perspective view of an exemplary portion 210 having one bend in the cannula, respectively. In some embodiments, the portion 210 may include a cannula 212 and a shape-memory element 214. The cannula 212 may include a bend 216. The single bend may help to ensure the shape-memory element 214 stays in-plane with the bend and adds an element of control. In some embodiments, the bend radius may be 2.75 mm. In some embodiments, the bend radius may be approximately 2.75 mm. In some embodiments, the shape-memory element 214 may have an open circular shape.

FIGS. 2E and 2F show a top view and a perspective view of an exemplary portion 220 having two bends in the cannula, respectively. In some embodiments, the portion 220 may include a cannula 222 and a shape-memory element 224. The cannula 222 may include a bend 226 and a bend 228. The additional proximal bend 228 may help to ensure the shape-memory element lays flat against the lens capsule (based on the geometry of the eye and typical incision location, this bend helps get the device past the iris and down fully on the lens capsule). In some embodiments, the proximal bend may be 11 degrees. In some embodiments, the proximal bend may be approximately 11 degrees.

FIGS. 3A to 3H illustrate exemplary variations of the shape-memory wire element(s), according to some embodiments.

FIG. 3A shows a perspective view of an exemplary shape-memory wire element 302, according to some embodiments. The shape-memory wire element 302 may have an essentially zero-pitch coil with minimal overlap. In some embodiments, the coil may include or may be a 370 degree “circle”. In some embodiments, the coil may include or may be an approximately 370 degree “circle”.

FIG. 3B shows a perspective view of an exemplary shape-memory wire element 304, according to some embodiments. The shape-memory wire element 304 may have an essentially zero-pitch coil with overlap greater than 360 degree “circle”, for example, approximately 730 degrees, approximately 1090 degrees, etc.

FIG. 3C shows a perspective view of an exemplary shape-memory wire element 306, according to some embodiments. The shape-memory wire element 306 may have a pitched coil with minimal overlap, for example, pitch of 1 mm, approximately 1 mm, or more or less. The coil may include or may be a 370 degree “circle”. In some embodiments, the coil may include or may be an approximately 370 degree “circle”.

FIG. 3D shows a perspective view of an exemplary shape-memory wire element 308, according to some embodiments. The shape-memory wire element 308 may have a pitched coil with overlap, for example, pitch of 1 mm, approximately 1 mm, or more or less; and for example, overlap greater than 360 degree “circle”, for example, approximately 730 degrees, approximately 1090 degrees, etc.

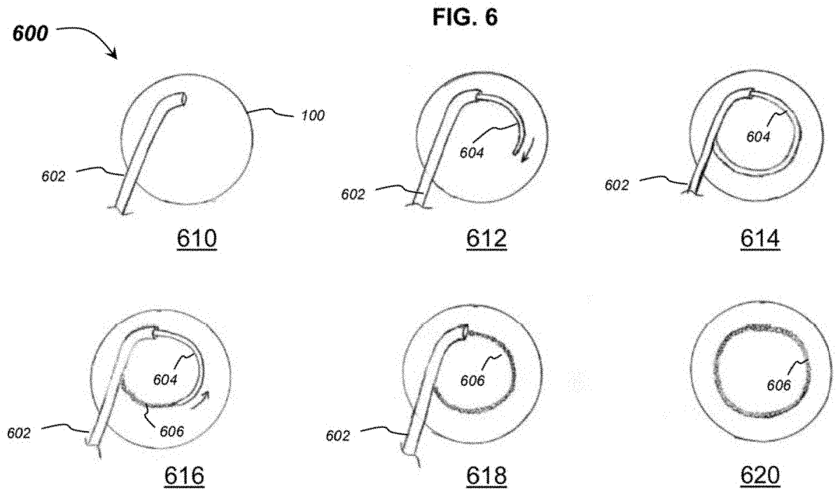

FIG. 3E shows a top view of an exemplary concentric configuration shape-memory wire element 310, according to some embodiments. FIG. 3E shows the concentric configuration-shape-memory element 310 coils under (for top-down approach, see FIG. 6 description) or under (for under-up approach) itself.

FIG. 3F shows a top view of an exemplary outward spiral configuration—shape-memory element 312 coils in-plane, according to some embodiments.

FIG. 3G shows a top view of an exemplary inward spiral configuration—shape-memory element 314 coils in-plane, according to some embodiments.

FIG. 3H shows a top view of an exemplary cross-arm configuration—shape-memory element 316, according to some embodiments.

It should be noted that the above configurations of the shape-memory wire element are examples and not meant to be limiting.

FIGS. 4A to 4AI illustrates exemplary variations of the shape-memory element(s), according to some embodiments.

FIGS. 4A to 4K illustrate cross-sectional views of various exemplary shape-memory elements, according to some embodiments. For example, the shape-memory element may include or may have a configuration as:

FIG. 4A: circular;

FIG. 4B: square;

FIG. 4C: triangular;

FIG. 4D: pie-shaped;

FIG. 4E: diamond;

FIG. 4F: rectangular;

FIG. 4G: thin foil;

FIG. 4H: multi-faceted (e.g., star-shaped);

FIG. 4I: oval;

FIG. 4J: crescent;

FIG. 4K: amorphous.

It should be noted that the above configurations of the shape-memory element are examples and not meant to be limiting.

FIGS. 4L to 4Q illustrate side views of various exemplary cutting/tearing elements coupled to or integral to the shape-memory elements, according to some embodiments. For example, the cutting/tearing element may include or may have a configuration as:

FIG. 4L: spear-shaped, with various angle A;

FIG. 4M: triangular-shaped, with various angle B;

FIG. 4N: trapezoidal, with various angle C;

FIG. 4O: triangular, with various angle D;

FIG. 4P: rectangular;

FIG. 4Q: trapezoidal/multi-faceted.

It should be noted that the above configurations of the shape-memory element are examples and not meant to be limiting.

FIGS. 4R to 4W illustrate side views of various exemplary shape-memory elements without the need for a coupled cutting/tearing element, but instead with various surface treatments, according to some embodiments. For example, the surface treatments may include or may be:

FIG. 4R: sandblasted or with media adhered to outer surface;

FIG. 4S: longitudinally grooved;

FIG. 4T: transversely grooved;

FIG. 4U: grooved at any angle;

FIG. 4V: cross-hatched in the longitudinal and transverse directions;

FIG. 4W: cross-hatched at any angle.

It should be noted that the above surface treatments are examples and not meant to be limiting.

FIGS. 4X to 4AC illustrate side views of various exemplary shape-memory elements without the need for a coupled cutting/tearing element, but instead with its end bent at various angles and variably sharpened, according to some embodiments. For example, the shape-memory element may include or may have a configuration as:

FIG. 4X: shape-memory element with its end bent greater than 90 degrees and tip sharpened to a point;

FIG. 4Y: shape-memory element with its end bent at 90 degrees and tip sharpened to a point;

FIG. 4Z: shape-memory element with its end bent less than 90 degrees and tip sharpened to a point;

FIG. 4AA: shape-memory element with its end bent greater than 90 degrees and tip flattened and sharpened;

FIG. 4AB: shape-memory element with its end bent at 90 degrees and tip flattened and sharpened;

FIG. 4AC: shape-memory element with its end bent less than 90 degree and tip flattened and sharpened.

It should be noted that the above configurations of the shape-memory are examples and not meant to be limiting.

FIGS. AD to 4AH illustrate side views of various exemplary shape-memory elements coupled to various rotating cutting elements, according to some embodiments. For example, the rotating cutting element may include or may have a configuration as:

FIG. 4AD: circular rotating cutting element;

FIG. 4AE: saw-like rotating cutting element;

FIG. 4AF: 3-pointed rotating cutting element;

FIG. 4AG: circular rotating cutting element with sharp protrusions (shown are 4, but can be at least 1);

FIG. 4AH: 4-pointed rotating cutting element.

It should be noted that the above configurations of the rotating cutting element are examples and not meant to be limiting

FIG. 4AI illustrates a side view of an exemplary shape-memory element that may be or may include a combination of any of the aforementioned elements/characteristics. In this example, shown is a rotating cutting element coupled to a shape-memory element that includes an end which is bent greater than 90 degrees and the tip sharpened to a point.

It should be noted that other configurations and combinations are also contemplated.

FIGS. 5A to 5K illustrate side views of additional exemplary, non-limiting variations of shape-memory elements, according to some embodiments. For example, the shape-memory element may include or may have a configuration with:

FIG. 5A: sharp hooked surface;

FIG. 5B: saw-tooth pattern;

FIG. 5C: saw-tooth pattern, or any cutting/tearing profile, that is adhered or joined to the surface of the shape-memory element;

FIG. 5D: at least one cutting element adhered or joined to its surface, the cutting elements being at any location/spacing along the shape-memory element (example shown has three cutting elements);

FIG. 5E: roughened surface at its distal end;

FIG. 5F: a cap at its distal end to protect other inner eye structures from potential piercing or damage. In some embodiments, the cap may be an actual cap/structural element, or a cap of adhesive (for example, preferably a biocompatible adhesive) that encapsulates the shape-memory element's distal end;

FIG. 5G: its distal end bent/folded back over itself tightly;

FIG. 5H: its distal end bent/rolled back over itself;

FIG. 5I: its distal end bent away from its longitudinal axis;

FIG. 5J: its distal end ground smooth;

FIG. 5K: a cap at its distal end, the cap comprising a roughened surface.

Turning to FIG. 6, an exemplary operation 600 of a shape-memory element device is illustrated, according to some embodiments. The method of operation 600 may be referred to as top-down method. At Step 610, a cannula 602 containing shape-memory element may be introduced into the anterior chamber of the eye 100. At Step 612, a shape-memory element 604 may be extended out of the cannula 602. At Step 614, after full extension, the shape-memory element 604 may be placed into contact with the lens capsule of the eye. Note that full extension of the shape-memory element can allow the operator to center the shape-memory element appropriately to the lens capsule.

At Step 616, while maintaining contact between the shape-memory element and the lens capsule, the shape-memory element 604 may be retracted, leaving either a visible path or groove 606 where it traveled (e.g., a circular template), a cut (e.g., a complete (360 degrees) or near complete (<360 degrees) cut to form the capsulotomy), or a tear (e.g., a complete (360 degrees) or near complete (<360 degrees) tear to form the capsulotomy).

In some alternative embodiments, a cutting element may be included on a portion of the shape-memory element, and during operation, only the cutting element portion touches the lens capsule.

At Step 618, the shape-memory element 604 may be fully retracted, leaving behind either a visible path or groove 606 where it traveled (e.g., a circular template), a cut (e.g., a complete (360 degrees) or near complete (<360 degrees) cut to form the capsulotomy), or a tear (e.g., a complete (360 degrees) or near complete (<360 degrees) tear to form the capsulotomy).

At Step 620, the cannula containing shape-memory element may be removed from the eye, leaving behind either a visible path or groove 606 where it traveled (e.g., a circular template), a cut (e.g., a complete (360 degrees) or near complete (<360 degrees) cut to form the capsulotomy), or a tear (e.g., a complete (360 degrees) or near complete (<360 degrees) tear to form the capsulotomy).

Variations of this method may include operation in the reverse, e.g., making a template, cut, or tear while extending the shape-memory element instead of during retraction. Another variation may include making a template, cut, or tear via both the extension and retraction movements of the shape-memory element. Yet another variation may include the use of an additional surgical tool to either grab and hold the capsule at its center, pierce through the capsule at its center, push down on the capsule at its center, or pull suction on the capsule at its center, all while the device per embodiments described herein is operated.

The device of the present disclosure may also be used in another operation method, which may be referred to as bottom-up method. This method may include similar steps to the top-down method, however upon initial extension of the shape-memory element, the shape-memory element may be directed to pierce through the capsule and extend underneath the capsule. Making the template, cut, or tear in the capsule can then commence via the same variations in method listed for the top-down method.

In some embodiments, contact of the shape-memory element to the lens capsule may be confirmed by the visible change in reflection/refraction of the lens capsule in the area immediately surrounding the shape-memory element; in other words, contact of the shape-memory element to the lens capsule may be confirmed by the visual presence of a “halo” that surrounds the shape-memory element.

In some embodiments, Purkinje images may be used for centering/aligning the shape-memory element, and thus the resultant capsulotomy template or capsulotomy.

As an example, a template-making device (meaning, a device that leaves a “template” circle for the surgeon to later follow after use of the device) includes a 0.008″ diameter nitinol wire sanded with a 36-grit sanding block (3 longitudinal strokes per wire side, with an additional 5 transverse strokes to create a cross-hatched pattern in the wire surface) formed into an approximately 370 degree circle with a 2.75 mm radius and with minimal pitch at its distal end via fixturing and heat treating at 500 degrees Celsius for 15 minutes followed by water quench. The formed nitinol wire is then placed into a steel cannula that has been bent at its distal end to match the radius of the formed nitinol wire, then operated as described herein

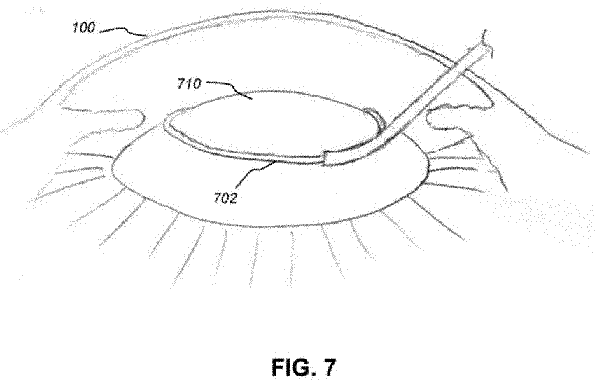

FIG. 7 illustrates an exemplary perspective view of a shape-memory element 702 being placed against lens capsule 710, according to some applications.

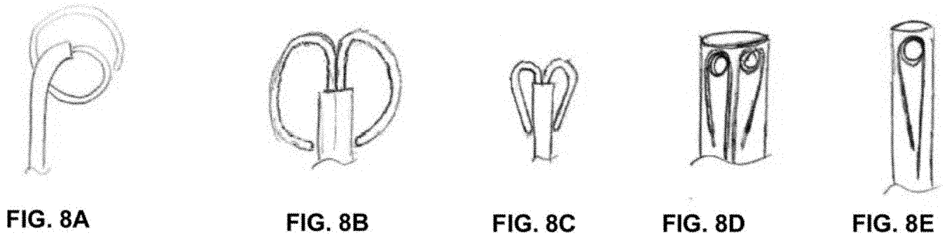

FIGS. 8A to 8E illustrate additional variations of wire-element devices, according to some embodiments.

FIG. 8A illustrates a top view of an exemplary outwardly expanding spiral shape-memory element, wherein the cannula is meant to be placed at the center of the desired capsulotomy (as opposed to other variants wherein the cannula is meant to be placed at the circumference of the desired capsulotomy).

FIG. 8B illustrates a top view of an exemplary dual extending/retracting shape-memory element configuration, wherein the shape memory elements create the capsulorhexis while being retracted; each element may be responsible for approximately 180 degrees of capsulotomy formation.

FIG. 8C illustrates a top view of an exemplary variation of a dual extending/retracting shape-memory element device, this variant with essentially linear “arms”; each element may be responsible for approximately 180 degrees of capsulotomy formation.

FIG. 8D illustrates a top view of another exemplary variation of a dual element device, wherein the elements may or may not be of a shape-memory material, and wherein upon retraction of an outer cannula or conversely extension of the dual elements beyond the cannula, the elements may create the capsulotomy via swinging around through release of pre-load (e.g., via a spring component integral to each element that can store potential energy); each element is responsible for approximately 180 degrees of capsulotomy formation.

FIG. 8E illustrates a top view of an exemplary single element device with characteristics similar to the dual element device, except the single element may be responsible for the full approximately 360-degree capsulotomy.

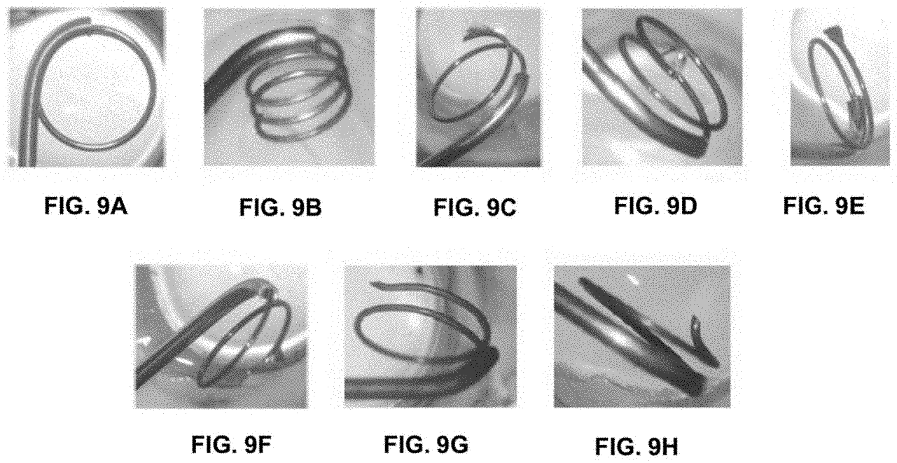

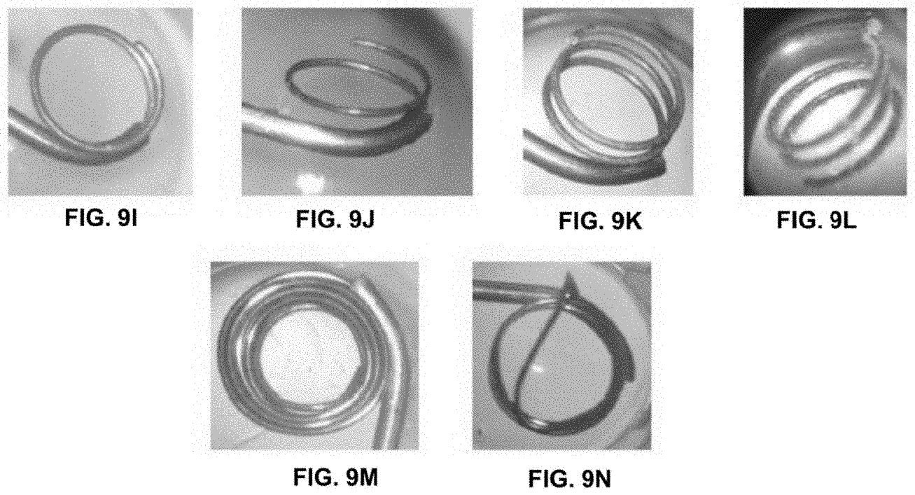

FIGS. 9A to 9N illustrate examples of shape-memory element devices reduced to practice.

FIG. 9A illustrates a top view of shape-memory element extended out of a cannula.

FIG. 9B illustrates a perspective view of a shape-memory element with a 1 mm pitch and overlap.

FIG. 9C illustrates a perspective view of a shape-memory element with a 1 mm pitch and spear diamond blade attached to its distal end.

FIG. 9D illustrates a perspective view of a shape-memory element with a 1 mm pitch and crescent diamond blade attached to its distal end.

FIG. 9E illustrates a perspective view of a shape-memory element with a minimal pitch and metal spear blade attached to its distal end.

FIG. 9F illustrates another perspective view of a shape-memory element with a 1 mm pitch and metal spear blade attached to its distal end.

FIG. 9G illustrates a perspective view of a shape-memory element with a 1 mm pitch and a flattened and sharpened distal end.

FIG. 9H illustrates another perspective view of a shape-memory element with a 1 mm pitch and a flattened and sharpened distal end.

FIG. 9I illustrates a perspective view of a shape-memory element with a minimal pitch and a roughened surface.

FIG. 9J illustrates a perspective view of a shape-memory element with a 1 mm pitch and a roughened surface.

FIG. 9K illustrates a perspective view of a shape-memory element with a 1 mm pitch, overlap and a roughened surface.

FIG. 9L illustrates a perspective view of a shape-memory element with a 1 mm pitch, overlap and abrasive compound adhered to its surface.

FIG. 9M illustrates a top view of a shape-memory element with a coiled inward and in-plane.

FIG. 9N illustrates a bottom view of a shape-memory element with a cross-arm and a metal spear blade adhered to its distal end.

FIGS. 10A to 10F illustrate various views of an exemplary capsulotomy shape-memory device 1000 reduced to practice. FIG. 10A illustrates the top view. FIG. 10B illustrates the right-side view. FIG. 10C illustrates the left-side view. FIG. 10D illustrates the bottom view. FIG. 10E illustrates the perspective view. FIG. 10F illustrates the partial exploded top view.

For example, FIG. 10E shows a cannula 1010, a shape-memory element 1012, a handle or outer shell 1014, and an actuator 1016. As shown, the shape-memory element 1012 is a wire element. The shape-memory element 1012 is housed within the cannula 1010. The cannula 1010 is housed within the outer shell 1014. The actuator 1016 is located on the outer shell and operably coupled to the shape-memory element 1012 to cause the element 1012 to extend and retract to and from the cannula's distal end.

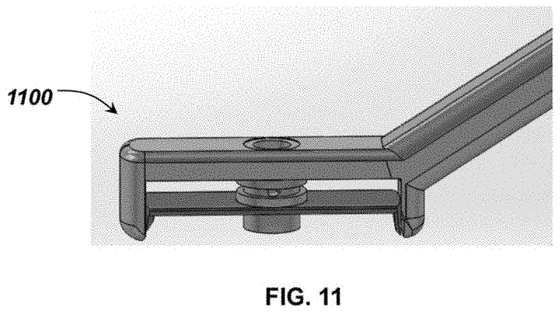

As illustrated in FIG. 11, in some embodiments, the rotating cutter (or cutting mechanism) may include one, two, or any number of cutting elements.

As used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many instances, entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic) intervening entities) and the indirect coupling of two entities (with one or more non-negligible intervening entities). Where entities are shown as being directly coupled together or described as coupled together without description of any intervening entity, it should be understood that those entities can be indirectly coupled together as well unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

What is claimed is:

1. A capsulotomy device comprising:
an outer shell;
a cannula housed within the outer shell, having proximal and distal ends;
a shape-memory filament housed within the cannula and extendable through the distal end of the cannula, the shape-memory filament having distal and proximal ends,
wherein the shape-memory filament has a bend at the shape-memory filament's distal end; and
an actuator located on the outer shell and operably coupled to the shape-memory filament to cause the filament to extend and retract to and from the cannula's distal end.

2. The capsulotomy device of claim 1, wherein the shape-memory filament has a circular shape.

3. The capsulotomy device of claim 1, wherein the shape-memory filament has one of square, triangular, pie, diamond, rectangular, thin foil, multi-faceted, oval, crescent, and amorphous shape.

4. The capsulotomy device of claim 1, wherein the tip of shape-memory filament's distal end is sharpened to a point.

5. The capsulotomy device of claim 1, wherein the tip of shape-memory filament's distal end is flattened and sharpened.

6. The capsulotomy device of claim 1, wherein the shape-memory filament includes a cutting surface.

7. The capsulotomy device of claim 1, wherein the shape-memory filament is coupled to a cutting element.

8. The capsulotomy device of claim 1, wherein the shape-memory filament is coupled to a rotating cutting element.

9. A capsulotomy device comprising:
a cannula having proximal and distal ends;
a shape-memory element housed within the cannula and extendable through the distal end of the cannula, the shape-memory element having distal and proximal ends,
wherein the shape memory wire has a bend at the shape memory wire's distal end; and
an actuator operably coupled to the shape memory wire to cause said wire to extend and retract to and from the cannula's distal end.

10. The capsulotomy device of claim 9, wherein the shape-memory element has a surface treatment.

11. The capsulotomy device of claim 10, wherein the surface treatment is one of sandblasted, grooved, and cross-hatched.

12. The capsulotomy device of claim 9, wherein the bend of shape-memory filament is greater than 90 degrees.

13. The capsulotomy device of claim 9, wherein the bend of shape-memory filament is less than 90 degrees.

14. The capsulotomy device of claim 9, wherein the bend of shape-memory filament is 90 degrees.

15. The capsulotomy device of claim 9, wherein the shape-memory element is coupled to a cutting element.

16. The capsulotomy device of claim 9, wherein the shape-memory element is coupled to a rotating cutting element.

17. A capsulotomy method comprising:
introducing a cannula containing a shape-memory element into an anterior chamber of an eye;
extending the shape-memory element out of the cannula;
placing the shape-memory element into contact with a lens capsule of the eye;
centering the shape-memory element on the lens capsule;
while maintaining contact between the shape-memory element and the lens capsule, retracting the shape-memory element into the cannula.
